(19) United States
(12) Patent Application Publication
(10) Pub. No.: US 2004/0267202 A1
(43) Pub. Date: Dec. 30, 2004

(54) Tearable hemostasis valve and splittable sheath

(76) Inventor: Daniel J. Potter, Stillwater, MN (US)

Correspondence Address: HEIMBECHER & ASSOCIATES, LLC., 390 UNION BLVD, SUITE 650, LAKEWOOD, CO, 80228-6512, US

(21) Appl. No.: 10/609,194

(22) Filed: Jun. 26, 2003

(51) Int. Cl. A61M 5/178

(52) U.S. Cl. 604/158; 604/160

(57) ABSTRACT

A tearable hemostasis valve for use with a splittable sheath. The hemostasis valve may include grip tabs, which may be pulled in generally opposing directions. The opposing forces tear open the valve body open along a score line. The tearable hemostasis valve includes a snap fit arrangement for mounting to a neck-and-hub arrangement of a splittable sheath. When the valve is pressed down onto the neck-and-hub arrangement, the snap fit arrangement deforms outwardly to permit the hub to pass through and into the cavity. After the hub passes through, the annular sidewall seats snugly around the neck. When an operation is complete, the tearable hemostasis valve may be torn open and discarded. The splittable introducer sheath may be torn open along its longitudinal axis by grasping opposing sheath wings and pulling in opposite directions. As the sheath is split, it may be withdrawn from a body and discarded.

### BACKGROUND OF INVENTION

a. Field of the Invention

The invention relates generally to the field of medical instruments, and more particularly to hemostasis valves for use during medical procedures.

b. Background Art

Several medical procedures require the introduction of one or more medical instruments into arteries or veins so that the medical instruments may be advanced to a body location requiring, for example, diagnosis or treatment. In one such procedure, known as the Seldinger procedure, a surgical opening or puncture is made in a vein or artery with a needle. A guide wire is then inserted through the lumen of the needle into the vein or artery. The needle is withdrawn, leaving the guide wire in place. A dilator is then inserted over the guide wire and used to dilate the puncture to a size that is sufficient to accommodate an introducer sheath. Once the sheath with at least one hemostasis valve is seated within the dilated puncture wound, maintaining relative hemostasis, the dilator and guide wire may be removed. With the sheath in place, the medical instruments (e.g., various types of catheters or leads) may be introduced through the hemostasis valve of the sheath and into the vein or artery, using the sheath as a conduit to prevent damage to the wall of the vein or artery, and into a region of the body to be diagnosed or treated.

In medical procedures where a pacemaker lead is inserted into a patient, a sheath is normally used to guide the pacemaker lead to the appropriate location. After the tip is secured in place and the lead attached to a pacemaker, the sheath must be removed. Because of the size of its lumen, the sheath cannot simply slip over the exterior end of the pacemaker lead as that end of the lead contains a connector coupling for connection to the pacemaker. Generally, a variety of splittable sheaths, capable of tearing along the longitudinal sheath axis, have been used to facilitate sheath removal without disturbing the aforementioned lead or connector.

One relatively simple method of limiting blood flow out of a sheath while a pacemaker lead is being introduced is for a physician to place his thumb over the exposed end of the sheath, or to squeeze or pinch the exposed end of the sheath. However, blood may still be lost and/or air introduced into the vessel when this method is used. In addition, the structure of many introducer sheaths requires the physician to hold the sheath securely while the sheath is in place in the vessel, thereby limiting the physician's ability to perform other medical procedures at the same time. Moreover, squeezing the exposed end of the sheath can deform or even break the sheath, making lead insertion difficult and increasing the likelihood of damage to the lead as it passes through the sheath. Further, even when holding or pinching the end of the sheath, the flow of blood out of the sheath is not entirely stopped.

As noted above, hemostasis valves have been attached to sheaths in order to limit or prevent blood flow out of the sheath. However, many such valves are also unable to fit over objects attached to the end of pacemaker leads or other items introduced into the sheath. Thus, conventional hemostasis valves may limit undesired blood flow, but reinstate many of the same problems previously discussed with respect to non-splittable sheaths.

Accordingly, there is a need for an improved hemostasis valve and sheath.

### SUMMARY OF INVENTION

Generally, an embodiment of the present invention takes the form of a tearable hemostasis valve. The tearable hemostasis valve may include a valve body, a pair of grip tabs attached to the valve body at first and second points, a score line disposed on the valve body between the first and second points, a membrane surrounded by the valve body, and a snap-fit arrangement attached to the valve body. The snap-fit arrangement generally mates with a tearable sheath. Alternative embodiments may omit the snap-fit arrangement and employ a sliding arrangement.

The valve may be torn open by pulling each of the grip tabs in generally opposing directions. The opposing forces tear open the valve body along the score line, and tear the membrane along a slit. In an alternative embodiment, multiple grip tabs may be used to facilitate splitting the valve.

In alternative embodiments, multiple membranes may be placed or formed within the valve body. These membranes may each have a slit therein. The slits may be angularly offset from one another. For example, the angle between a slit in a first membrane and a slit in a second membrane may be thirty to forty-five degrees, or greater or lesser if desired. The multiple membranes and slits assist in minimizing blood loss by offsetting the tears in the slits, thus creating an overall smaller overlapping hole, when viewed through all membranes.

The tearable hemostasis valve may be mounted to a splittable sheath. Generally, the splittable sheath includes a neck-and-hub arrangement for mounting the hemostasis valve. The snap fit arrangement typically includes an annular sidewall defining an opening and a cavity within the valve body that is slightly larger in cross-section than the opening. When the valve is pressed down onto the neck-and-hub arrangement, the annular sidewall deforms outwardly to permit the hub to pass through and into the cavity. After the hub passes through, the annular sidewall seats snugly around the neck. The combination of the snap fit arrangement and neck-and-hub arrangement creates a seal sufficiently snug to prevent blood from exiting the sheath.

When an operation is complete, the tearable hemostasis valve may be torn open, as described above, and discarded. Further, the splittable introducer sheath may include a pair of opposed sheath wings generally attached to the sheath body. By grasping the sheath wings and pulling in opposite directions, the sheath may be split along its longitudinal axis. As the sheath is split, it may be withdrawn from a body and discarded.

In some cases, a surgeon may find it necessary to continue a medical procedure after the valve has been torn. In such a case, the tearable hemostasis valve may be re-closed about the sheath. The valve body may have sufficient shape memory to at least partially return to its initial shape (i.e., the shape of the valve before being torn). Further, when re-closed the valve body may force the torn portions of the membrane together, thus providing at least a partial seal around the sheath.

### SUMMARY OF DRAWINGS

FIG. 1 depicts an isometric view of a tearable hemostasis valve in accordance with a first embodiment of the present invention.

FIG. 2 depicts a top-down view of the tearable hemostasis valve of FIG. 1.

FIG. 3 depicts a cross-sectional view taken along line 3-3 of FIG. 2.

FIG. 4 depicts a cross-sectional view, similar to that shown in FIG. 3, of an alternate embodiment of the tearable hemostasis valve depicted in FIGS. 1 and 2.

FIG. 5 depicts a cross-sectional view of the tearable hemostasis valve depicted in FIGS. 1-3, mounted to a first embodiment of a splittable introducer sheath.

FIG. 6 depicts an isometric view of the tearable hemostasis valve depicted in FIGS. 1-3, mounted to the first embodiment of a splittable introducer sheath.

FIG. 7 depicts an isometric view of the tearable hemostasis valve depicted in FIG. 6 being removed from the first embodiment of a splittable introducer sheath.

FIG. 8 depicts an isometric view of the tearable hemostasis valve depicted in FIGS. 6 and 7 being separated from the first embodiment of a splittable introducer sheath.

FIG. 9 depicts an isometric view of the splittable introducer sheath depicted in FIGS. 6-8 being removed from about a lead.

FIG. 10 depicts an isometric view of a second embodiment of a tearable hemostasis valve incorporating dual membranes.

FIG. 11 depicts a top-down view of the tearable hemostasis valve of FIG. 10.

FIG. 12 depicts a cross-sectional view of the tearable hemostasis valve of FIG. 10, taken along line 12-12 of FIG. 11.

### DETAILED DESCRIPTION OF PREFERRED EMBODIMENTS

Generally, one embodiment of the present invention takes the form of a hemostasis valve capable of being torn or split open (“tearable”). The hemostasis valve may be configured to act as an introducer cap, clamping onto or otherwise attaching to an introducer sheath suitable for placement at least partially in a blood vessel or other portion of a body. Objects foreign to a body, such as a pacemaker lead, may be introduced into the body by feeding through the aforementioned sheath until a proper point inside a patient's body is reached.

The hemostasis valve may include a seal membrane having a slit or other opening through it. When the valve is placed atop the sheath, the membrane prevents blood from flowing out through the sheath due to a pressure differential between the blood vessel interior and the atmosphere. Further, the aforementioned foreign object may be introduced into the sheath through the membrane slit of the valve, thus permitting the object to be moved along the sheath and into the body while maintaining the seal created by the hemostasis valve.

Although reference is made herein to “slits” in the membranes, it should be understood that the term “slit” must not necessarily connote an opening extending entirely through the membrane. As used herein, “slit” is intended to embrace scores or depressions that may not extend through a membrane.

When an operation is complete, the introducer sheath and hemostasis valve may be split apart in order to permit the sheath and valve to be removed from the implanted lead or other foreign object without requiring either the sheath or valve to slide over the free end of the foreign object or another object inserted therethrough. The split sheath and valve may be removed without interfering with either the foreign object or attached object. This permits removal of both the sheath and valve without disturbing delicate items or instrumentation, such as a pacemaker lead. The tearable hemostasis valve may be split or torn in such a manner that it may be removed from about the foreign object either simultaneously with or separately from the splittable introducer sheath.

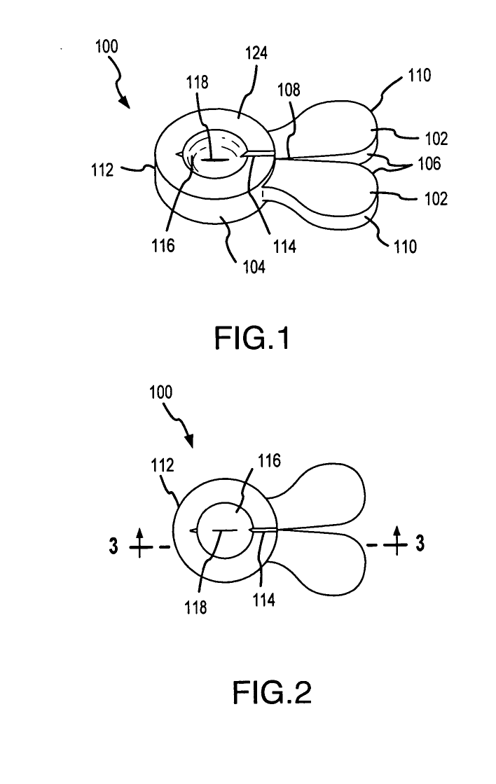

FIG. 1 shows an isometric view of a first embodiment of a tearable hemostasis valve 100. Generally, a pair of grip tabs 102 extend outwardly from a portion of a body sidewall 104. The interior edges 106 of each grip tab define an angular space 108, which tapers from the outermost edges 110 of the grip tab to a point at which the interior edges of each grip tab contact the body sidewall 104. Typically, the interior edge of each grip tab contacts the body sidewall at substantially the same point, although alternative embodiments may slightly offset the grip tabs from one another along the body sidewall.

Extending from the narrowest portion of the angular space 108 across the top of the valve body 112 is a score line 114. Generally, the score line 114 extends at least partially downwardly through the body 112. The score line may have either a V-shaped, U-shaped, or rectangular-shaped cross section, and has a shape and depth through the body sufficient to assist in splitting or tearing the body when the grip tabs 102 are firmly grasped and moved in opposite directions. The score line may extend onto a portion of the valve body across the membrane 116 effectively forming a “notch” or second score line 120. This second score line 120 may be placed on the valve body directly opposite the first score line 114. The second score line may extend across the entirety of the opposing. This second score line 120, or notch, may assist in separating or opening the hemostasis valve 100 along its lateral axis by providing a path for tearing, snapping, or otherwise separating the valve body 112.

The valve body 112 generally includes at least one valve sidewall 104. The valve sidewall runs parallel to a longitudinal axis of the hemostasis valve 100, and defines an outer surface of the valve body. In the embodiment of FIG. 1, the valve body 112 is generally cylindrical, and accordingly has a single valve sidewall 104. Alternative embodiments may vary the shape of the valve body 112, making it square, triangular, elliptical, oblate, and so forth in cross-section taken through the valve sidewall, or may curve, taper, stair-step or otherwise shape the sidewall. The first 114 and second 120 score lines mentioned above may extend downwardly along portions of the sidewall 104.

In addition to the sidewall 104, the valve body is defined by a valve top surface, which in the present embodiment takes the form of an annular ring 124. Alternative embodiments may employ a flat, continuous top surface having no holes or depressions formed therein. Generally, the first score line 114 (and second score line 120, if present) are formed on the annular ring 124. A valve membrane 116 is typically located in the recess formed by the ring 124.

The valve membrane 116 is secured to the hemostasis valve body 112. In one embodiment, the membrane and valve body may be integrally formed as a single, continuous piece. Alternatively, the membrane 116 and body 112 may be discretely formed, with the membrane securely affixed to the body at some point during construction of the tearable hemostasis valve. For example, the membrane may be heat sealed, attached by a solvent, clamped within, or sonically welded to a portion of the body. Alternately, the membrane 116 and valve body 112 may be co-extruded, in which case the two elements may be made of the same or differing materials.

Similarly, the valve body 112 may be overmolded over the membrane 116, thus affixing the membrane within the valve 100. Effectively, the membrane may be molded from a lower durometer material and the valve body from a higher durometer material. During overmolding, the membrane is generally extruded and molded after the valve body, but this molding procedure may be reversed.

Regardless of the manner in which the membrane 116 is affixed to the valve body, the joinder between membrane and body is sufficiently close to avoid impairing the sealing properties of the hemostasis valve. A slit 118 may be provided in the membrane to permit a lead or other item to pass through the membrane and into the introducer sheath. Generally, the slit 118 is sized to snugly fit around the lead, in order to maintain a good seal and thus minimize blood flow through the sheath and beyond the membrane.

The exact configuration of the membrane slit 118 may vary from embodiment to embodiment. The slit 118 may be a straight line, as shown in FIG. 1, Y-shaped with straight or curved lines forming the parts of the Y, semicircular, two intersecting lines, and so forth. Similarly, the exact thickness, shape, and configuration of the membrane 116 may vary widely. In one embodiment, for example, the membrane may be cylindrical, while in another it may have a tapered or wasp-waisted middle section. In yet another embodiment, the membrane 116 may taper from its exterior surfaces towards its middle. Any and all membrane configurations known to those skilled in the art may be used with the present tearable hemostasis valve 100.

Alternatively, the membrane slit 118 may be replaced by a score. A lead may be pushed through the score in much the same manner as previously described with respect to the slit. The act of pushing the lead against the score partially tears the membrane 116 along the score, thus opening a path for introduction of the lead into the disposable sheath. By using a score instead of a slit 118, the possibility that the membrane opening (i.e., the slit or score) is larger than the cross section of the lead is minimized. In yet another embodiment, the membrane may be sufficiently thin that a lead may be forced through it without requiring either a slit or a score. Accordingly, when discussing the membrane 116 all references to a slit 118 should be understood to include a score, and vice versa.

Further, in any of these embodiments, the membrane 116 may be self-sealing in order to minimize any leakage introduce between the valve 100 interior and atmosphere when a foreign object is passed through the membrane 116. That is, the membrane 116 may be formed from a material having some ability to seal a torn edge or punctured portion, typically by adhering or chemically bonding torn or punctured portions to one another.

As previously mentioned, the valve body 112 and membrane 116 may be manufactured either as a single piece or as two connecting pieces. The membrane 116 are typically manufactured from a plastic or elastomeric material, such as silicone rubber, latex rubber or a foamed rubber of 20-60 durometer. While the valve body 112 may also be formed from a plastic or elastomeric material, it typically is formed from a stiffer material (i.e., one having a higher durometer) than that used to form the membrane, such as a hard plastic or high density polypropylene. Alternative embodiments of the valve 100 and membrane 116 may be manufactured from a plastic material sufficiently deformable and resilient to allow the membrane to be pierced by a pacemaker lead or other foreign object, while retaining its overall shape, and while providing a generally airtight seal around the lead. In alternative embodiments, the membrane and body may be fabricated from two materials having different elastic properties. For example, the membrane 116 may be created from a material having a relatively low durometer (or material hardness), while the valve body 112 may be manufactured from a material having a higher durometer. By using a low durometer material for the membrane and a higher durometer material for the body, consistent hemostasis and easy tearing of the valve are both provided.

FIG. 2 depicts a top down view of the tearable hemostasis valve 100 of FIG. 1. As can be seen in FIG. 2, the slit 118 in the membrane 116 is generally aligned with the score line 114 through the body 112. Thus, when the valve body is torn along the score line, the membrane will generally split along the slit.

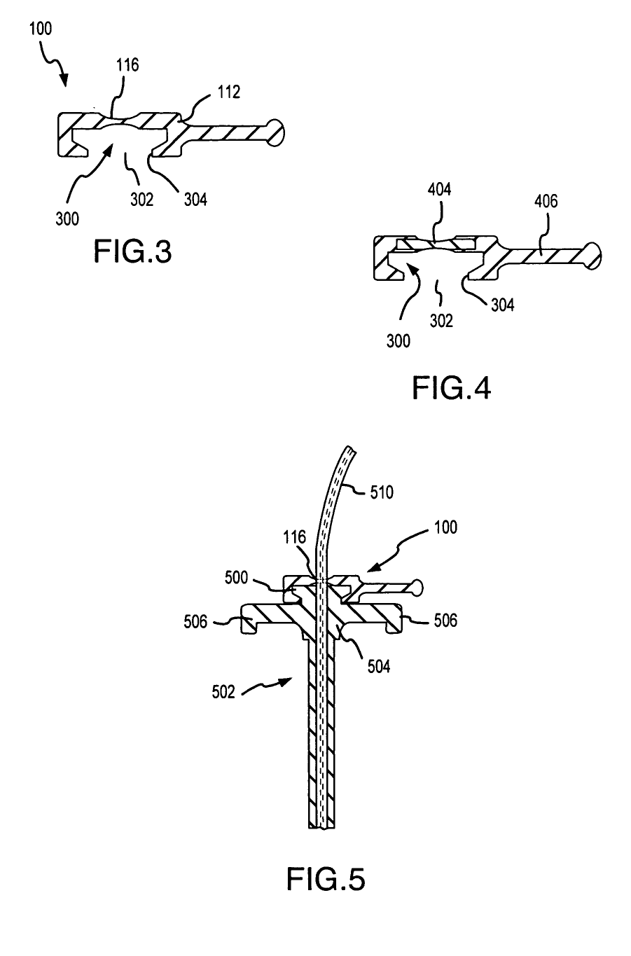

FIG. 3 depicts a cross-sectional view, taken along line 3-3 of FIG. 2, of the present embodiment of the tearable hemostasis valve 100. In this embodiment, the membrane 116 and valve body 112 are manufactured as a single, continuous piece, and accordingly are generally manufactured of the same material. By contrast, the embodiment shown in FIG. 4 (which is a cross-sectional view similar to the view depicted in FIG. 3) has a membrane 404 mounted or formed within a valve body 406. In this embodiment, the membrane 404 and valve body 406 are manufactured of two separate materials. The valve body 406 and membrane 404 may be joined together at some point during manufacture or operation of the valve, or may be co-extruded from two materials during manufacture. When the valve 406 and membrane 404 are co-extruded, they generally form a unitary element, with the membrane joined to the valve by the intermingling and bonding of the two materials along the union between membrane and valve body. Accordingly, the valve body and membrane shown in FIG. 4 may be manufactured from two different materials, or may be manufactured from the same material.

As shown in both FIG. 3 and FIG. 4, a cavity 300 is defined within the valve interior. An opening 302 through the bottom of the hemostasis valve 100 extends into the cavity. An annular ring or sidewall 304 encircles and defines the opening. Generally, both the opening 302 and cavity 300 are circular when viewed in top-down cross section (as opposed to the side view cross section shown in FIGS. 4A and 4B), although the shape of the opening and cavity may vary in other embodiments. Further, the diameter of the opening is typically smaller than that of the cavity. Collectively, the annular sidewall 304, opening 302, and cavity 300 comprise one example of a snap fit arrangement, although alternative embodiments may use different snap fit arrangements. As shown in FIG. 5, the cavity 300 is sized to snugly receive a hub 500 of an introducer sheath 502.

Instead of the neck-and-hub arrangement discussed immediately above, an alternative embodiment of the present invention may include a slot or opening located along the valve body sidewall 104. This arrangement may permit the tearable hemostasis valve 100 to be slid onto the neck and hub, rather than snapped on or otherwise pushed downward onto the neck and hub. Generally, the slot is sized so that the hub is slightly compressed by the valve body 112, or fits inside the slot sufficiently snugly to form a seal between the sheath and valve 100.

A modified, splittable introducer sheath 502 may be used with the tearable hemostasis valve 100 to create a seal, thus preventing blood flow up the sheath 502, through the valve, and out of a patient. The seal may also prevent air bubbles from forming within the sheath. The introducer sheath may include a flared hub 500 mounted on a neck 504. The neck, in turn, is affixed to one or more sheath wings 506, as shown in FIG. 5. Typically, the neck 504 is smaller than the hub 500, when viewed in cross-section taken parallel to the transverse axis of the sheath. The sheath wings extend generally perpendicularly from the body 508 of the splittable introducer sheath. A lead 510 may be introduced into the sheath 502 interior by passing it through the membrane 116.

The sheath hub 500 is sized to be snugly received within the hemostasis valve cavity 300. Similarly, the sheath neck 504 is sized to come into contact with the vertical sidewall 304 of the valve opening 302 when the valve is mounted to the sheath 502.

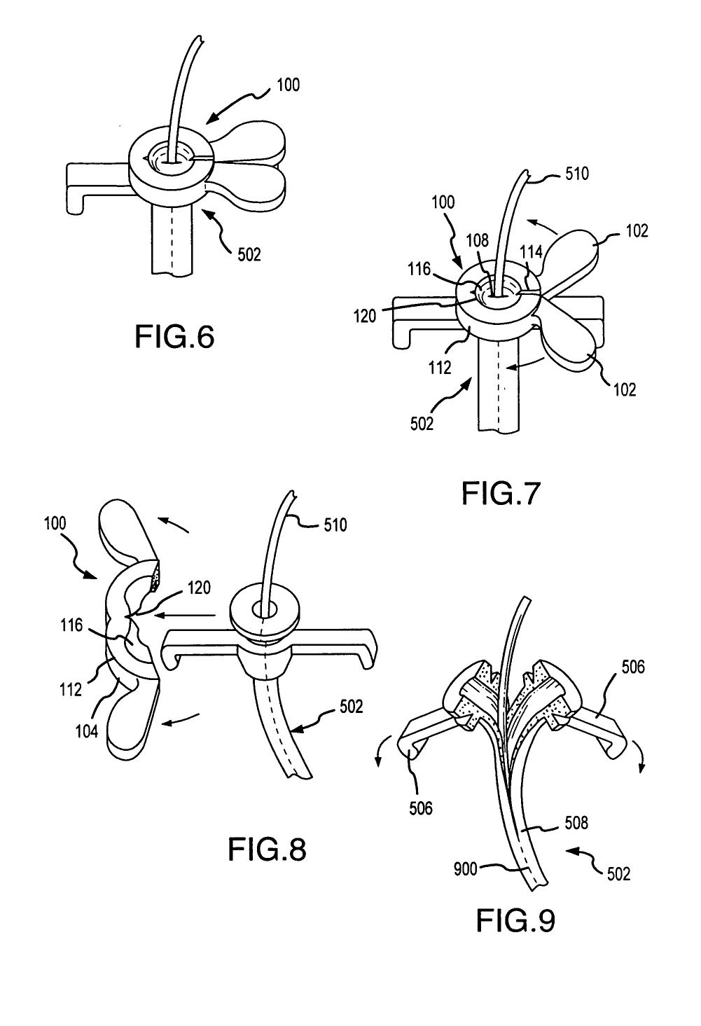

When the sheath 502 has been inserted into a patient's body (or prior thereto), the tearable hemostasis valve 100 may be snap fitted to the neck-and-hub arrangement of the sheath. Either the valve body 112 (including the annular sidewall 304) or the neck-and-hub arrangement of the introducer sheath is sufficiently flexible to permit the neck-and-hub to mate with the valve cavity 300 and opening 302. Accordingly, the valve 100 may snap fit onto the introducer sheath. Both the valve and neck-and-hub arrangement are sized in such a manner as to create a seal sufficient to prevent introduction of an unacceptable amount of air from outside the valve/sheath arrangement into the sheath interior, or blood flowing in the opposite direction. Although not shown in FIG. 5, the introducer sheath 502 or valve may include a side port lumen and tubing. FIG. 6 shows an isometric view of the hemostasis valve 100 mounted atop the splittable sheath 502.

The tear direction of the valve 100 is generally perpendicular to that of the introducer sheath 502. By placing these tear directions at right angles to one another, accidental simultaneous tearing of both the valve 100 and sheath 502 may be avoided. Thus, if the valve is prematurely removed from the sheath, the sheath may remain intact. In such event, only the valve 100 need be replaced, rather than both the valve and sheath 502.

As previously mentioned, when an operation is complete, the valve 100 and sheath 502 may be quickly removed without disturbing either the object within the sheath or requiring the valve and sheath to pass over any item connected to the object. This is accomplished by tearing at least a portion of the valve 100 and splitting the body 508 of the sheath. This process is shown in FIGS. 7-9. When the sheath 502 is initially removed and the seal created by the sheath eliminated, blood may freely flow through the sheath and out of a patient's body. Accordingly, the less time taken to remove the sheath 502 and hemostasis valve 100, the less blood loss experienced by the patient.

Removal of the tearable hemostasis valve 100 and introducer sheath 502 begins in FIG. 7. Here, each grip tab 102 is pulled away from the opposite grip tab in the direction shown by the arrows. These opposing forces pull the valve body 112 apart along the score line 114. As the valve body breaks, the membrane 116 tears in generally the same direction. In the present embodiment, tearing of the membrane occurs along the membrane slit 118. The presence of the slit may reduce the amount of force required to tear the elastic membrane.

Additionally, the second score line 120 may assist in opening or tearing the valve, as shown in FIG. 8. Generally, the second score line 120 provides a tear path along the side of the valve body 112 opposite the grip tabs 102, fulfilling the same function as and yielding similar results to the first score line 114.

As also depicted in FIG. 8, once the membrane tear reaches the slit 118, the valve 100 may be removed from the hub and neck arrangement of the splittable sheath 502 without disturbing the pacemaker lead. The lead 510 slides out of the torn end of the valve 100, all the while remaining securely inside the sheath 502. Generally, removal of the valve 100 from the sheath 502 (as shown in FIG. 8) does not disturb or move the lead 510. The portion of the valve body 112 originally opposite the first score line 114 (i.e., the portion of the valve body upon which the second score line 120 is formed) may or may not also be torn. As shown in FIG. 8, this portion of the valve body 112 may simply deform but otherwise remain intact. When the valve body is made of sufficiently resilient material or material having a shape memory, the membrane 116 may be replaced or repaired, the valve bent or otherwise returned to its original shape, and the valve reused.

For example, a surgeon may remove the valve 100 from the sheath 502, then find it desirable to replace the valve about the sheath to continue a procedure. The valve body 112 may be placed with the sheath 502 located in the cavity and the lead or device passing through the now-torn membrane 116 (i.e., passing through the torn membrane slit 118). Once the valve 100 is positioned, the surgeon may apply force to bend or return the valve body 112 to its original shape, typically by grasping the opposing grip tabs 102 or opposing portions of the valve body sidewall 104 and forcing the tabs/portions towards one another. Since the membrane 116 is affixed to the valve body 112, this action also forces the torn edges of the membrane slit 118 towards one another, providing a temporary seal.

Once the valve 100 is removed from the introducer sheath 502, the opposing sheath wings 506 may be grasped and pulled in opposite directions, as shown in FIG. 9. This force causes the sheath 502 to split along its longitudinal axis. The sheath body 508, including the neck 500 and hub 504, may be scored 900 or grooved along its longitudinal axis to assist in tearing the sheath. As the sheath 502 is torn, it may be pulled out of the patient's body. Once the sheath 502 is completely free of the body, the wound made to insert the sheath may be treated.

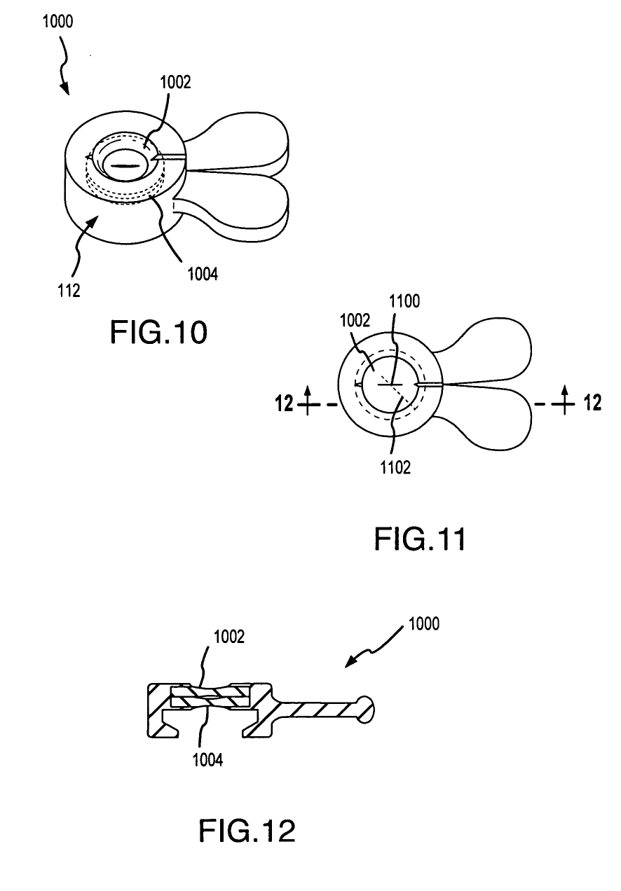

Generally, the tearable hemostasis valve 100 embodiment discussed above includes a single membrane. Alternative embodiments, however, may make use of multiple membranes in a single valve body 112. For example, the valve 1000 shown in FIG. 10 has two membranes 1002, 1004, generally located one atop the other. The membranes are typically of the same material, but may be fabricated from differing materials in alternative embodiments.

Each membrane may have a slit formed therein to facilitate passage of a medical device (such as a lead) through the membrane. Each membrane is typically attached to or held within the valve body 112, as discussed above. The slits 1100, 1102 may be angularly offset from one another, as depicted to better effect in FIG. 11. It should be understood that slit 1102 is formed in the lower membrane 1004, which is blocked from view in FIG. 11 by the upper membrane 1002. Further, although the upper and lower membranes are generally illustrated in FIGS. 10 and 11 as being the same size, they may vary in size in alternative embodiments.

By rotating the slits 1100, 1102 with respect to one another, blood loss through the slits may be minimized. As a device passes through or punctures a slit, the opening in the slit may extend beyond the outer edges of the device, creating a gap space (not shown) through which blood may flow out of the sheath 502 and valve 1000 arrangement. Since the slit 1100 in the first membrane 1002 and slit 1102 in the second membrane 1004 are angularly offset, so too are any gap spaces created in each slit. Accordingly, the blood outflow path defined by the gap spaces is minimized, because each gap space at least partially abuts a portion of the adjacent membrane.

Typically, the angular offset of the slit may be any angle up to and including forty-five degrees, although alternative embodiments may offset the slits by an even greater angle. The offset angle between slits 1100, 1102 generally allows the second membrane 1004 to tear or open as the valve body 112 and first membrane 1002 split. Accordingly, the angular offset between slits 1100, 1102 may vary depending on the location of the grip tabs 102 with respect to one another along the valve body.

Further, one or both slits 1100, 1102 may extend to the edge of the membranes 1002, 1004. For example, the second slit 1102 is shown extending to the membrane edge in FIG. 11. In this configuration, both slits need not be torn when the valve 1000 is removed from a sheath 502. Typically, one slit 1100 is formed in the middle of a membrane 1002 and does not extend to a membrane edge, while the second slit 1102 does extend to the edge of the second membrane 1004. Accordingly, the second membrane 1004 does not offer as much resistance to tearing or opening the valve 1000 as it would if the slit were only in the center of the membrane, thus reducing the force necessary to open the valve.

As with other embodiments discussed above, both membranes 1002, 1004 may be co-extruded with the valve body 112, overmolded by the body, or affixed thereto during creation of the valve. Generally, and as depicted in cross-section in FIG. 12, the first 1002 and second 1004 membranes abut one another within the valve body 112. That is, generally the bottom of the first membrane 1002 overlies and abuts the top of the second membrane 1004. In alternative embodiments, a space may be present between membranes 1002, 1004, or a portion of the valve body 112 may at least partially separate the membranes. Further, it should be noted that a small space 1200 is shown in FIG. 12 between membranes. This space typically results from variances in the membrane thickness. Alternative embodiments may employ relatively uniformly thick membranes 1002, 1004, thus eliminating or reducing this space.

As will be recognized by those skilled in the art from the foregoing description of embodiments of the invention, numerous variations on the described embodiments may be made without departing from the spirit and scope of the invention. For example, an alternative embodiment may employ three or more membranes, rather than the one or two discussed herein. Yet another embodiment may employ membranes of differing sizes, materials, or durometers. Further, while the present invention has been described in the context of specific embodiments and processes, such descriptions are by way of example and not limitation. Accordingly, the proper scope of the present invention is specified by the following claims and not by the preceding examples.

What is claimed is:

1. A tearable hemostasis valve, comprising:
a valve body;
a first grip tab attached to said valve body at a first point;
a second grip tab attached to said valve body at a second point;
a score line disposed on said valve body between said first and second points;
a first membrane disposed within said valve body; and
a snap-fit arrangement attached to said valve body.

2. The valve of claim 1, wherein said first membrane is bonded to said valve body.

3. The valve of claim 1, wherein said first membrane is integrally formed with said valve body.

4. The valve of claim 3, wherein said first point and said second point are the same.

5. The valve of claim 4, wherein:
said first membrane comprises a material of a first durometer; and
said valve body comprises a material of a second durometer, said second durometer higher than said first durometer.

6. The valve of claim 5, further comprising a score in said first membrane.

7. The valve of claim 5, further comprising a second membrane disposed within said valve body parallel to said first membrane.

8. The valve of claim 7, wherein said first and second membranes are self-sealing.

9. The valve of claim 8, wherein said snap-fit arrangement comprises:
a cavity disposed in said valve body; and
an annular sidewall defining an opening in communication with said cavity.

10. The valve of claim 9, wherein said annular sidewall is flexible.

11. The valve of claim 9, wherein said opening is located below said cavity.

12. The valve of claim 9, wherein said opening is located along one side of said cavity.

13. A method for removing a sheath from a patient's body, comprising:
splitting a hemostasis valve attached to said sheath along a lateral axis of said valve;
splitting said sheath along a longitudinal axis of said sheath; and
removing said valve from said sheath; and
pulling said sheath from said patient's body while splitting said sheath.

14. The method of claim 13,wherein the step of removing said valve from said sheath comprises sliding said valve away from said sheath along a split formed along said lateral axis.

15. The method of claim 14, wherein the step of splitting said sheath along a longitudinal axis of said sheath comprises:
exerting force on at least one sheath wing away from said longitudinal axis; and
tearing said sheath along a score line parallel to said longitudinal axis.

16. A splittable sheath, comprising:
a flexible body defining a hollow cavity therein;
means for splitting said sheath operably connected to said flexible body at least one sheath wing operably connected to said means for splitting;
a neck affixed to said flexible body and having a first diameter; and
a hub affixed to said neck, said hub having a second diameter greater than said first diameter.

17. The splittable sheath of claim 16, wherein said neck and hub define a pathway from said cavity to an exterior of said sheath.

18. The splittable sheath of claim 17, wherein said means for splitting comprises at least one score line.

19. The splittable sheath of claim 18, wherein said at least one score line extends parallel to a longitudinal axis of said sheath.

20. The splittable sheath of claim 19, wherein said at least one score line extends along the entirety of said neck, hub, and body.
